(19) United States
(12) Patent Application Publication
(10) Pub. No.: US 2020/0179331 A1
(43) Pub. Date: Jun. 11, 2020

(54) Peptide-Artesunate Conjugates as Targeted Anti-Cancer Agents

(71) Applicant: University of Massachusetts, Boston, MA (US)

(72) Inventor: Maolin Guo, Dartmouth, MA (US)

(21) Appl. No.: 16/627,239

(22) PCT Filed: Jun. 29, 2018
(86) PCT No.: PCT/US2018/040330

§ 371 Date: Dec. 27, 2019

Related U.S. Application Data
(60) Provisional application No. 62/527,290, filed on Jun. 30, 2017.

(51) Int. Cl. A61K 31/357; A61K 47/64; A61K 45/06; A61P 35/00

(52) U.S. Cl. CPC A61K 31/357; A61P 35/00; A61K 45/06; A61K 47/64

(57) ABSTRACT

The disclosure provides new anti-cancer compositions that include a Her2 peptide that targets the Her2 receptor and comprises amino acid sequence GSGKCCYSL (SEQ ID NO:1); and a covalently-linked cytotoxic agent including artemisinin (Art) or a derivative thereof linked to the peptide. Biological assays have demonstrated that the Art-Her2 peptide conjugates described herein show excellent selective cytotoxic activity towards Her2-positive cancers, such as colon cancer, compared to normal colon cells.

### CROSS-REFERENCE TO RELATED APPLICATIONS

This application is an international application and claims priority to U.S. Provisional Patent Application Ser. No. 62/527,290, filed on Jun. 30, 2017. The present application hereby incorporates the entire contents of the provisional application by reference.

### TECHNICAL FIELD

This invention relates to anti-cancer agents, and more particularly to peptide-artesunate conjugates as targeted anti-cancer agents.

### BACKGROUND

Cancer has now become one of the top two leading causes of human deaths in US and other western countries. There is high demand for more effective and innovative anti-cancer drugs. Many anticancer drugs are highly cytotoxic agents and are often associated with severe adverse side effects in cancer treatments, as the active cytotoxic agents, which are designed to kill cancer cells also damage healthy normal cells and tissues. Over the past few decades, researchers have been actively working on the development of targeted anti-cancer drugs designed to delivers an active anti-cancer agent directly to the cancer cells without harming normal healthy cells or tissues. Targeted cancer chemotherapy is a strategy designed to target cytotoxic agents specifically near or into cancer cells, like a “magic bullet,” killing or blocking the growth of cancer cells while, at the same time, limiting damage to normal healthy cells. These targeted drugs thus tend to have less side effects compared to standard chemotherapeutic drugs.

The most successful targeted therapeutics are antibody-drug conjugates (ADCs), which represent an innovative therapeutic application that combines, via a linker, the unique, high specificity of monoclonal antibodies that are tumor-specific, with the potent cell killing activity of cytotoxic small molecule drugs (payloads). In linking monoclonal antibodies with cytotoxic agents, though remarkably challenging, scientists have been able to optimize the features of both components, successfully creating highly effective cancer therapies that benefit patients with hematologic and solid tumors. While antibody-drug conjugates have shown promise and have achieved notable success in cancer therapy, this approach has also met with limitations in efficacy for the widespread use of these novel agents. One of the most common issues limiting the effectiveness of antibody-drug conjugates is poor cell permeability and low tumor permeation by the large antibody molecules (˜150 kDa). A second issue is that the payload that an antibody, which is a large protein molecule, can carry is relative small, i.e., about 1/500 of its own weight.

### SUMMARY

The targeted anti-cancer drug delivery methods and compositions described herein improve therapeutic efficiency and reduce potential toxic side effects by limiting damage to normal cells. To overcome the limitations of known methods and compositions, the new methods and compositions include new, targeted anti-cancer therapeutics using a small peptide-drug Her2-artemisinin derivative conjugate, which is potent and selective and is uniquely designed to target Her2 positive tumors, accumulate their payload in these tumors, and cause death of the cells in these tumors.

The Her2-Art conjugates described herein include a peptide ligand that is highly selective in targeting, and dependent on, the tumor cell-surface Her2 receptor, which is overexpressed on the surface of cancer cells in a variety of cancers, including, for example, breast, lung, liver, colon, prostate, bladder, cervix, endometrium, germ cell, glioblastoma, head and neck, ovarian, pancreas, salivary duct, and gastric cancer. By carefully designing and creating the novel conjugate with a potent cell-killing payload of artesunate, one can minimize payload dissociation and achieve desired cancer killing while avoiding toxicity to normal cells.

The small Her2 peptide in the conjugate has been designed to contain only 9 amino acids with a MW of 917 Da. This is much smaller than an antibody, which is usually a few hundred times larger. The small size of Her2-Art enables effective penetration and distribution into the tumor tissue and associated extracellular matrix, resulting in better penetrating and targeting of tumors. The ligand's targeting ability also enables specific binding to Her2-positive tumor cells, bringing the potent, cell-killing payload inside the cancer cells for high efficacy.

In one aspect, the present disclosure features anti-cancer compositions that include a Her2 peptide that targets the Her2 receptor and comprises amino acid sequence GSGKCCYSL (SEQ ID NO:1); and a cytotoxic agent comprising artemisinin (Art) or a derivative thereof linked to the peptide. The cytotoxic agent can be or include one or more of artesunate and dihydroartemisinin.

In some implementations, the Her2 peptide and cytotoxic agent are chemically linked via peptide conjugation chemistry. In some embodiments, the compositions include two or more Her2 peptides. In certain implementations, an amine functional group of an N-terminus of the Her2 peptide is chemically linked to a carbonyl group of artemisinin or derivative thereof. For example, the artemisinin derivative can be artesunate.

In various implementations, the compositions can further include one or more supplementary active agents selected from the group consisting of adriamycin, cyclophosphamide, taxotere, vinblastine, dacarbazine, etoposide, vincristine, procarbazine, predniscone, cisplatin, 5-fluorouracil, and gemcitabine.

In another aspect, the present disclosure features compositions as described herein for use in treating a Her2-positive cancer. In this aspect, the Her2-positive cancer can be selected from the group consisting of breast, lung, liver, colon, prostate, bladder, cervix, endometrium, germ cell, glioblastoma, head and neck, ovarian, pancreas, salivary duct, and gastric cancer.

In another aspect, the present disclosure features methods of inhibiting growth of Her2-positive cells in a subject, such as a human or animal subject (e.g., mouse, rat, rabbit, dog, cat, horse, cow, sheep, pig, monkey, and ape). The methods include identifying a subject with Her2-positive cells; and administering to the subject an effective amount of a composition including a Her2 peptide that targets the Her2 receptor and comprises amino acid sequence GSGKCCYSL (SEQ ID NO:1); and a cytotoxic agent linked to the peptide, wherein the cytotoxic agent comprises artemisinin (Art) or a derivative thereof.

In these methods, the cytotoxic agent can be or include one or more of artesunate and dihydroartemisinin, and the compositions can further include one or more supplementary active agents selected from the group consisting of adriamycin, cyclophosphamide, taxotere, vinblastine, dacarbazine, etoposide, vincristine, procarbazine, predniscone, cisplatin, 5-fluorouracil, and gemcitabine.

Compared to antibody-drug conjugates, the peptide conjugates described herein can carry a large payload, i.e., about one third of their weight (˜170 fold increase compared to that of antibody conjugates). Moreover, the small Her2 peptide can be produced by automation and the linkage process described herein is a simple one-step synthetic chemical procedure, which is inexpensive. This methodology offers significant economic advantages to produce the new conjugates, because it is much simpler and quicker than the methods required to prepare antibody drug conjugates.

Advantages of the new compositions and methods include one or more of the following:

1) Artesunate is competent to generate Fe²⁺⁻dependent free radicals in vitro and exhibit cytotoxicity against cancer cells grown in culture.

2) The potent cytotoxicity of artesunate against several human cell lines and the enhancing effect of iron ions were demonstrated. Artesunate inhibits the proliferation of HCT-116 cancer cells with IC50 ˜0.63 μM. [3-{4, 5-dimethylthiazol-2-yl)-2, 5-diphenyltetrazolium bromide (MTT) assays determined IC50 of artesunate against normal colon CCD-18 cells and cancerous colon Caco-2 cells to be ˜5.6 μM and ˜0.97 μM, respectively.

3) Confocal imaging with the Fe sensor determined endogenous labile Fe²⁺ concentration in the cancerous colon Caco-2 cells is significantly higher than that in normal colon CCD-18 cells, correlating the increased cytotoxicity of Art-Her2 conjugate against cancerous cells with higher intracellular Fe²⁺ levels.

4) A novel drug-peptide conjugate, an Art-Her2 conjugate that is composed of artesunate and a small Her2 peptide, is chemically synthesized, purified by high pressure liquid chromatography (HPLC) and characterized by UV-vis and ultra performance liquid chromatography—mass spectrometer (UPLC-MS).

5) The cytotoxic effects of the Art-Her2 conjugate are higher against Her2-positive cancer cells compared to Her2-negative cells. The Art-Her2 conjugate has been shown to selectively kill cancerous HER-positive Caco-2 cells with a potent IC50 ˜15 μM. However, normal CCD-18 cells are not susceptible to Art-Her2 and kept health in all the concentrations tested (up to 100 μM). Thus, the Art-Her2 conjugate selectively kills Her2-positive cancer cells and may be promising to represent a novel class of anticancer drug for targeted cancer therapy for Her2-positive cancers.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIGS. 1A to 1C are representations of the chemical structures of Artemisinin (ART or Art) (FIG. 1A) and ART-derived compounds: Artesunate (FIG. 1B), and Dihydroartemisinin (FIG. 1C).

FIG. 2 is a schematic of the potential Fe²⁺⁻dependent chemical rearrangements of ART and ART-derived compounds result [27].

FIG. 3 shows absorbance spectra of 100 μM Artesunate in dimethyl sulfoxide (DMSO), 100 μM HER2 targeting peptide (GSGKCCYSL; SEQ ID NO:1) in H₂O, and 100 μM Artesunate-Her2 targeting peptide conjugate in dimethylformamide (DMF).

FIG. 4 is a representative HPLC chromatogram of the Artesunate-Her2 targeting peptide (Art-HER2) conjugate. The mobile phase consisted of H₂O (solvent A) and acetonitrile (solvent B). The separation was performed on a semi-preparative XTerra RP18 column (250×10 mm) with a gradient of 0% solvent B to 100% solvent B over 60 minutes at a flow rate of 8 mL/min with detection wavelength 254 nm.

FIG. 5 is a graphic representation of LC-MS data of ART-HER2 conjugate in DMF.

FIG. 6 is a graphic representation of data from deoxyribose degradation assays performed with various Fe2+ concentrations (5, 10, 15, 20, 25 μM) and initiated either with 200 μM Artesunate or 200 μM hydrogen peroxide (H₂O₂). The absorbance at 532 nm reflects the level of monoaldehyde-thiobarbituric acid (TBA) complex formed in each assay; higher absorbance values at 532 nm indirectly reflect higher levels of hydroxyl radicals or peroxyl radicals.

FIG. 7 is a graphic representation of a concentration-response curve of DCF fluorescence after 30 minute exposure of 10 μM of 2′-7′-dichlorodihydrofluorescein diacetate (DCFH)-DA to various concentrations of Fe²⁺ (5, 10, 15, 20, 25 μM) and initiated with either 20 μM H₂O₂ or 20 μM artesunate.

FIG. 8 is a representation of a series of microscopic photographs of trypan blue stained B16-F10 cells: (a) untreated cells with no artesunate or no Fe²⁺ (Control), (b) cells treated with 4 μM Fe²⁺ for 24 h, (c) cells treated with 200 μM artesunate for 24 hours, and (d) cells treated with 4 μM Fe²⁺ and 200 μM artesunate for 24 h.

FIG. 9 is a representation of a series of microscopic photographs of Trypan blue stained HCT116 cells (human colon carcinoma cell line): (a) untreated cells with no artesunate and no Fe²⁺ (Control), (b) cells treated with 4 μM Fe²⁺ for 24 h, (c) cells treated with 20 μM artesunate for 24 h, (d) cells treated with 4 μM Fe²⁺ and 20 μM artesunate for 24 hours.

FIG. 10 is a graphic representation of dose-response analysis of artesunate toxicity against HCT116 cells using the sulforhodamine B colorimetric (SRB) assay in a 96-well format.

FIG. 11 is a graphic representation of dose-response analysis of artesunate toxicity against a Caco-2 cell line using the MTT assay in a 96-well format.

FIG. 12 is a graphic representation of dose-response analysis of artesunate toxicity against a CCD-18 cell line using the MTT assay in a 96-well format.

FIG. 13 is a graphic representation of dose-response analysis of Art-Her 2 conjugate toxicity against a Caco-2 cell line using the MTT assay in a 96-well format.

FIG. 14 is a graphic representation of dose-response analysis of Art-Her 2 conjugate toxicity against a CCD-18 cell line using the MTT assay in a 96-well format.

Like reference symbols in the various drawings indicate like elements.

### DETAILED DESCRIPTION

To overcome the limitations of antibody-drug conjugates, we have developed a novel class of potent and selective targeted drug conjugates by linking the high-targeting capability of a small Her2 peptide (0.9 kDa), with a potent cytotoxic agent, e.g., artensunate or other artemisinin derivatives. This innovative approach resulted in the creation of an ART-HER2 conjugate as a novel targeted anti-cancer agent. The ART-HER2 has high affinity for the cell surface receptor Her2, which is over-expressed in many types of cancer cells, and results in increased ART delivery to the HER2-positive cancer cells and thus, the ART-HER2 conjugate exhibits increased cytotoxicity against HER2-positive cancer cells. Compared to the antibody-drug conjugates, this peptide conjugate can carry a large payload, i.e., about one third of its own weight, which is an approximately 170-fold increase compared to that of antibody drug conjugates. Moreover, the small Her2 peptide can be produced by automation using a new linkage procedure in a one-step chemical synthesis, which is a lost cost method.

Biological assays described in the Examples below demonstrate that the ART-HER2 conjugates show excellent selective cytotoxic activity towards Her2-positive colon cancer cells compared to normal colon cells, demonstrating that it is a targeted anticancer agent against Her2-positive colon cancers.

### Artemisinin, Derivatives, and Analogs

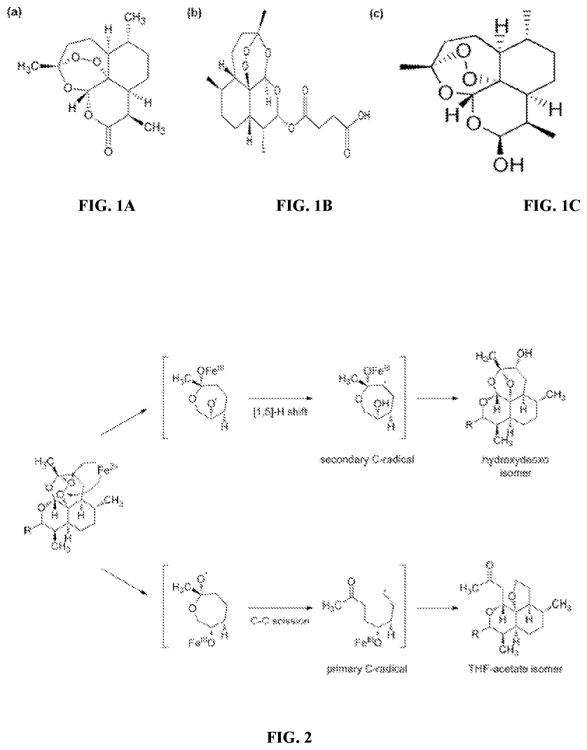

Artemisinin (ART or Art) is a small molecule that is the active principle of the Chinese medicinal herb Artemisia annua L. Artemisinin (FIG. 1A) and its derivatives and analogs such as artesunate (FIG. 1B) and dihydroartemisinin (FIG. 1C) are sesquiterpene lactone peroxides containing an endoperoxide moiety, which forms free radicals when it reacts with ferrous ions through a Fenton-like reaction. Artesunate and other derivatives are known as an anti-malarial drug (C19H28O8) and are semisynthetic and water-soluble, and are taken orally or administered by intramuscular or intravenous injection [1-6]. ART and ART-derived drugs function to reduce and clear malaria infections by reacting with Fe⁺ derived from hemoglobin in red blood cells infected with Plasmodium falciparum malaria to generate lethal reactive radical species that damage cellular components and ultimately trigger cell death, reducing numbers of P. falciparum in patients [7, 8-12]. FIG. 2 shows the proposed mechanism of Fe²⁺ activation of the endoperoxide group in ART and ART-derived compounds [27].

### Human Epidermal Growth Factor Receptor 2 (HER2)

Human epidermal growth factor receptor 2 (HER2 or Her2), also known as ErbB2 or HER2/neu, encoded by a proto-oncogene HER2/neu. HER2 is over-expressed on cell membranes of many types of cancer cells, including breast, lung, liver, colon, prostate, bladder, cervix, endometrium, germ cell, glioblastoma, head and o neck, ovarian, pancreas, salivary duct and gastric cancer; this HER2 overexpression has been termed HER2-positive [33]. Patients with HER2-positive cancer cells typically have more aggressive cancer, often with increased instances of metastasis and evolved resistance to chemotherapy regimens. The discovery of HER2 as a cancer-specific factor spurred the development of HER2 targeting moieties including monoclonal antibodies (mAbs) against HER2, humanized recombinant mAbs against HER2, and peptides targeting HER2.

### ART-HER2 Conjugates

The present invention concerns chimeric agents comprising a peptide targeting HER2 linked to ART or ART derivatives, referred to herein as ART-HER2 conjugates. These conjugates are prepared by covalently linking a chemically synthesized HER2-targeting peptide, GSGKCCYSL (SEQ ID NO:1), to artesunate. We present below in the Examples results of biological assays that indicate the ART-HER2 conjugate exhibits increased toxicity against HER2-positive cancer cells, specifically Caco-2 cells grown in culture, compared to the action of ART or ART-derivatives alone. Thus, the new ART-HER2 conjugate is a novel therapeutic agent with increased cytotoxicity against HER2-positive cancer cells.

The ART-HER2 conjugates were synthesized via a peptide conjugation chemistry: the HER2 targeting peptide [GSG-KCCYSL (CONH2] has an amine functional group present at the N-terminus and is competent for acid-amine conjugation to the carbonyl group of Artesunate. In brief, Artesunatem, EDC1, and HOBt were dissolved in DMF and incubated with HER2 targeting peptide [GSG-KCCYSL (CONH2)] (53 mg, 0.058 mmol) at room temperature under N2 atmosphere. The solvent was concentrated by rotary evaporator and the resulting Artensunate-HER2 targeting peptide (ART-HER2) conjugate was purified by semi-preparative reverse-phase HPLC. To verify ART-HER2 purity, mass spectrometry analysis was performed on the pooled product; TOF-MS ES+: calctd 1283.49, found 1283.58 corresponding to the expected m/z ratio of the ART-HER2 conjugate.

### Methods of Treatment

The methods described herein include methods for the treatment of disorders associated with Her2-positive cells. In some embodiments, the disorder is cancer. Generally, the methods include administering a therapeutically effective amount of Art-Her2 conjugate as described herein, to a subject who is in need of, or who has been determined to be in need of, such treatment.

As used in this context, to “treat” means to ameliorate at least one symptom of the disorder associated with aberrant proliferation, gene expression, signaling, translation, and/or secretion of factors. Often, the presence of Her2-positive cancer cells results in a poor prognosis for cancer patients; thus, the goal of treatment as described herein is a reduction in Her2-positive cells and a reduction in the number of cancer cells in the patient. Administration of a therapeutically effective amount of a compound described herein for the treatment of a condition associated with Her2-positive cells will result in decreased proliferation or reduction in total cell numbers of Her2-positive cells.

The Art-Her2 are useful in the treatment of disorders associated with abnormal apoptotic or differentiative processes, e.g., cellular proliferative disorders or cellular differentiative disorders, e.g., cancer, e.g., by producing an active or passive immunity. Examples of cellular proliferative and/or differentiative disorders include cancer, e.g., carcinoma, sarcoma, metastatic disorders or hematopoietic neoplastic disorders, e.g., leukemia. A metastatic tumor can arise from a multitude of primary tumor types, including, but not limited to, those of prostate, colon, lung, breast, and liver origin.

As used herein, the terms “cancer,” “hyperproliferative,” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

### Pharmaceutical Compositions and Methods of Administration

The methods described herein include the use of pharmaceutical compositions comprising Art-Her2 as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., adriamycin, cyclophosphamide, taxotere, vinblastine, dacarbazine, etoposide, vincristine, procarbazine, predniscone, cisplatin, 5-fluorouracil, or gemcitabine.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel®, or corn starch; a lubricant such as magnesium stearate or Sterotes®; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the therapeutic compounds are prepared with carriers that will protect the therapeutic compounds against rapid elimination from the body, such as a controlled release formulation, including albumin adducts, implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

In one embodiment, the therapeutic compounds are prepared with carriers that will protect the therapeutic compounds against rapid elimination from the body, such as a controlled release formulation, including albumin adducts, implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. Nanoparticles (1 to 1,000 nm) and microparticles (1 to 1,000 μm), e.g., nanospheres and microspheres and nanocapsules and microcapsules, can also be used. These can be prepared according to methods known to those skilled in the art, for example, as described in U.S. Pat. No. 4,522,811; Bourges et al., Ocular drug delivery targeting the retina and retinal pigment epithelium using polylactide nanoparticles. Invest Opth Vis Sci 44:3562-9 (2003); Bourges et al., Intraocular implants for extended drug delivery: therapeutic applications. Adv Drug Deliv Rev 58:1182-1202 (2006); Ghate et al., Ocular drug delivery. Expert Opin Drug Deliv 3:275-87 (2006); and Short, Safety Evaluation of Ocular Drug Delivery Formulations: Techniques and Practical Considerations. Toxicol Pathol 36(1):49-62 (2008).

### Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### EXAMPLES

The invention is further described in the following examples, which do not limit the scope of the invention described in the claims.

### Example 1. Synthesis of ART-HER2 Conjugate

The present Example describes the chemical synthesis and purification of an ART-HER2 conjugate.

### Materials

Artesunate, 1-ethyl-3-(3-dimethylaminopropyl)carbodiimide hydrochloride (EDC1) and 1-hydroxybenzotriazole (HOBt), and HER2 peptide [GSG-KCCYSL (CONH₂)] used were purchased commercially and used without further purification.

### Instruments and Methods

ESI-MS analyses were performed using a Waters ACQUITY® UPLC Q-TOF mass spectrometer. High Performance Liquid Chromatography (HPLC) separation was performed using an Agela Technologies chromatography instrument. The mobile phase consisted of H₂O with 0.1% TFA (Solvent A) and acetonitrile with 0.1% TFA (Solvent B). The HPLC separation was performed on a semi-preparative XTerra® RP18 column (250×10 mm) with a gradient of 0% Solvent B to 100% B over 60 minutes at a flow rate of 8 mL/min.

### Results

The Art-Her2 conjugates were synthesized via a peptide conjugation chemistry (Scheme 1). Briefly, Artesunate (20 mg, 0.053 mmol), EDC1 (7.7 mg, 0.04 mmol) and HOBt (6 mg, 0.036 mmol) were dissolved in DMF. Following stirring of reaction for 1 h, HER2 targeting peptide [GSG-KCCYSL(CONH₂)(SEQ ID NO:1)] (53 mg, 0.058 mmol) was added to the mixture and stirred at room temperature for 24 hours under N₂ atmosphere. The solvent was concentrated by rotary evaporator and the resulting Artensunate-HER2 targeting peptide (ART-HER2) conjugate was purified by semi-preparative reverse-phase HPLC. The fractions corresponding to ART-HER2 from multiple runs were collected and pooled. The ART-HER2 conjugate was purified by semi-preparative reverse-phase HPLC (FIG. 4) with 26-percent product yield of 26-percent.

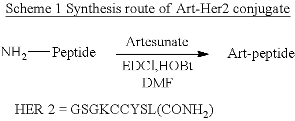

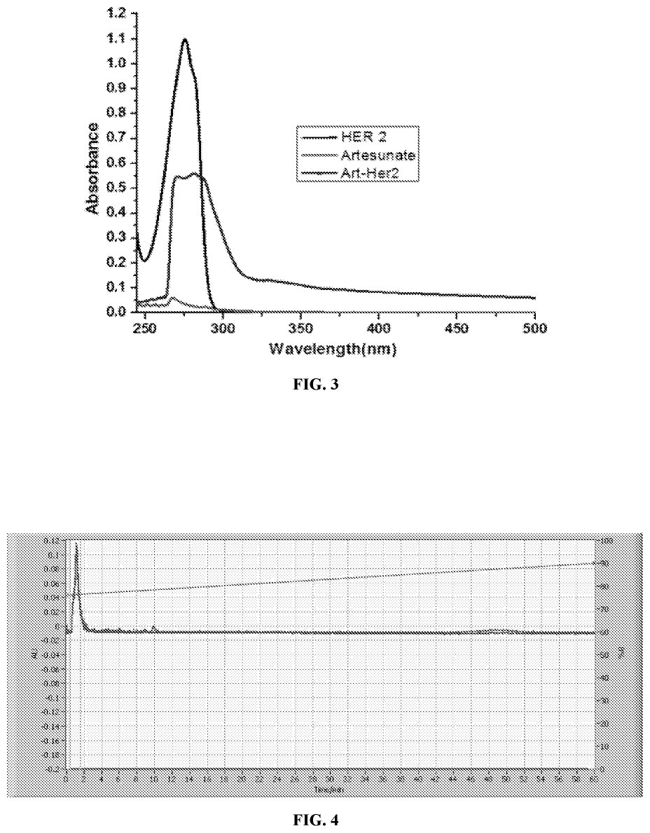

To verify ART-HER2 purity, mass spectrometry analysis was performed on the pooled product; TOF-MS ES+: calctd 1283.49, found 1283.58 corresponding to the expected m/z ratio of the ART-HER2 conjugate. The ART-HER2 conjugate was characterized by UV-Vis (FIG. 3) and verified by ESI-MS (FIG. 5). FIG. 3 shows the absorption spectra of Artesunate (100 μM, the line at the bottom), HER 2 (100 μM, the line at top) and Art-Her2 conjugate (100 μM, the line at middle) in DMSO, H₂O, DMF respectively. FIG. 4 shows the HPLC profile of the ART-HER2 conjugate. The mobile phase consisted of H₂O (solvent A) and acetonitrile (solvent B). The separation was performed on a semi-preparative XTerra RP18 column (250×10 mm) with a gradient of 0% to 100% B in 60 min at a flow rate of 8 mL/min with detection wavelength 254 nm. FIG. 5 shows the LC-MS data of Art-Her2 conjugate in DMF.

The presented chemical evaluations of our ART-HER2 conjugate reveal that the conjugate is highly pure.

### Example 2. Characterize Competency of ART-HER2 Conjugates In Vitro to Form Free Radical Species Conditional on Fe²⁺

Experiments were performed to determine if the ART-HER2 conjugate (described in Example 1) reacts with Fe2⁺ in vitro to result in formation of free radical species.

### Materials and Methods

### Deoxyribose Degradation Assays

This assay quantifies the formation of hydroxyl radicals or peroxyl radicals [39]. The assay was carried out following a reported procedure [40]. Briefly, 500 μL reaction volumes containing 10 mM 2-deoxyribose, 100 μM ascorbic acid, and various concentrations of Fe2+ were prepared. Degradation of 2-deoxyribose was initiated either by addition of 200 μM of hydrogen peroxide or 200 μM of Artesunate. The reaction was allowed to run for 10 minutes and then stopped by the addition of 500 μL of 10% (w/v) trichloroacetic acid (TCA) followed by 0.5 mL 1% 2-thiobarbituric acid (TBA). After heating at 80° C. for 15 minutes, the absorbance at 532 nm was measured.

### Dichlorofluorescein Assay

The assay was carried out following a reported procedure either in presence of hydrogen peroxide, Artesunate with and without Fe2+, and ART-HER2 conjugate with and without Fe2+[43]. In this assay non-fluorescent DCFH-DA (2′, 7′-Dichlorofluorescin diacetate treated with methanol and NaOH, as described in reference 43) is converted to a fluorescent derivative, known as dichlorofluorescein (DCF), in presence of an oxidizing agent (e.g. reactive oxygen species). By quantifying the concentration of fluorescent DCF, the concentration of oxidizing agent can be determined [42].

### Results

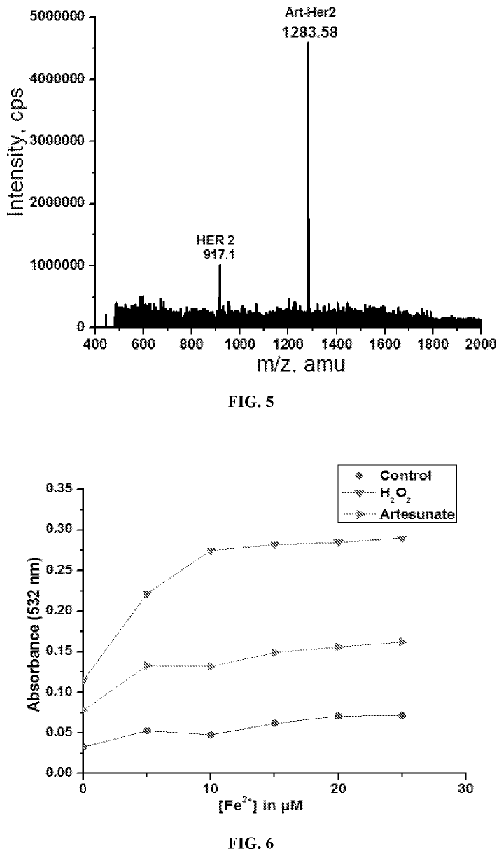

To measure the concentration of free radical species generated by Artesunate reacting with Fe²⁺, 2-deoxyribose degradation assays were formed (FIG. 6). Degradation of 2-deoxyribose by action of hydrogen peroxide is shown as a positive control and 2-deoxyribose alone is shown as a negative control (FIG. 6). The results show that Artesunate reacts with Fe²⁺ to generate free radical species that are sufficient to degrade 2-deoxyribose and the amount of 2-deoxyribose degradation increases with increasing concentrations of Fe⁺.

FIG. 6 shows the absorbance of monoaldehyde-TBA complex at 532 nm from reactions containing various Fe²⁺ concentrations (5, 10, 15, 20, 25 μM) and initiated either with 200 μM Artesunate or 200 μM hydrogen peroxide.

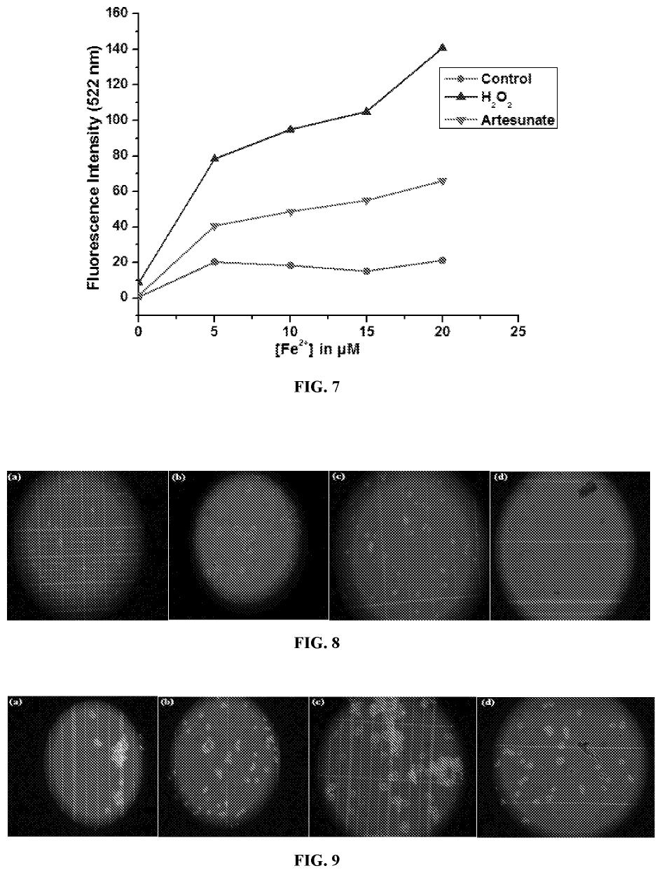

Formation of free radical species from the reaction of Artesunate with Fe⁺ was additionally measured by dichlorofluorescein assays. Oxidation of 2′, 7′-Dichlorofluorescin diacetate (DCFH-DA) by reactive oxygen species generates dichlorofluorescein, which is fluorescent (˜520 nm). As seen in FIG. 7, both H₂O₂ and Artesunate showed Fe²⁺-dependent activation of dichlorofluorescein fluorescence, demonstrating reactive oxygen species formed between the interactions of artesunate with Fe²⁺. In particular, FIG. 7 shows the concentration-response curve of DCF fluorescence after 30 min exposure of 10 μM of DCFH-DA to various concentrations of Fe²⁺ (5, 10, 15, 20, 25 μM) and either H₂O₂ (20 μM) or artesunate (20 μM).

The presented results of the 2-deoxyribose degradation and dichlorofluorescein assays indicate that the ART component of our ART-HER2 conjugate is competent to form free radical species in presence of Fe⁺. Additionally, the results show that the level of free radical species generated by our ART-HER2 conjugate increases in presence of increasing levels of Fe⁺. Thus, our prepared ART-HER2 conjugate possesses the expected chemical composition and the expected ability to react with Fe²⁺ and form free radical species.

### Example 3. Cytotoxicity of Artesunate and ART-HER2 Conjugate Against Cancer Cells Grown in Culture With and Without Extracellular Fe²⁺

Experiments were performed to assess cytotoxicity of Artesunate and the ART-HER2 conjugate (described in Example 1) against cancer cells grown in culture.

### Materials and Methods

### Cell Culture

Mouse B16-F10 melanoma cancer cells and human HCT116 colon cancer cells were grown in culture respectively in DMEM medium or McCoy's 5 A medium supplemented with 10% fetal bovine serum and 5% antibiotic in a 5% CO2 atmosphere at 37° C. Cultures were divided 1:2 every 48 h to an approximate cell density of 1.2 million cells per ml and used for experiments after 24 hours of growth in culture.

### Confocal Imaging

A Zeiss LSM 710 laser-scanning confocal microscope system with a 40× oil-immersion objective lens was used for cell imaging experiments. To image ferrous iron molecules in cells, a Fe2+ sensor NIRh-Fret was used with excitation wavelengths of the laser at 405 nm and 633 nm and emissions were collected over the range 420-700 nm and 650-850 nm, respectively.

### Sulforhodamine B (SRB) Colorimetric Assay

The SRB assay determines cell density based on measurement of cellular protein content. The principle underlying the SRB assay is SRB, a pink aminoxanthane dye, binds proteins from cells fixed by TCA and SRB binding is stoichiometric given each SRB has two sulfonic groups that can react with basic amino acids in proteins and SRB dissociates under basic conditions. As the binding of SRB is stoichiometric, the amount of dye extracted from stained cells is directly proportional to cell mass [48]. The assay was carried out following a reported procedure [41]. In brief, cell monolayers were fixed by 10% (wt/vol) trichloroacetic acid and stained for 30 minutes. Excess dye was removed by washing cells with 1% (vol/vol) acetic acid. The protein-bound dye was dissolved in 10 mM Tris base solution and optical density at 510 nm was measured using a microplate reader.

### MTT Assay

The assay was carried out following a reported procedure [45]. In brief, approximately 1,000 to 5,000 cells were plated per well in 96-well plate. The plates were incubated at 37° C. for 48 or 72 hours in a humidified, 5% CO2 atmosphere. Cells were incubated either with Artesunate or with ART-HER2 conjugate at various concentrations. To measure mitochondrial activity, a metric for cell viability, 15 μL of [3-{4, 5-dimethylthiazol-2-yl)-2, 5-diphenyltetrazolium bromide (MTT) was added to each well and incubated at 37° C. for 4 hours in a humidified, 5% CO2 atmosphere. After incubation, 100 μl of the Solubilization Solution/Stop Mix to each well was added. The blue formazan product formed by reduction of MTT by mitochondrial dehydrogenase in viable cells was then measured by absorbance at 570 nm [44].

### Results

To explore the cytotoxicity of Artesunate against cancer cells and role of extracellular Fe⁺ concentration, mouse B16-F10 skin melanoma cancer cells were incubated with 200 μM Artesunate and 0 or 4 μM extracellular Fe²⁺ for 24 hours. The number of viable cells was determined by staining with Trypan blue, a dye that selectively stains dead cells blue, and quantifying microscopic images to determine percentage of viable cells. As shown in FIG. 8, untreated cells and cells treated with extracellular Fe²⁺ are highly viable with most cells remaining unstained. In contrast, Artesunate-treated cells showed a higher fraction of dead cells (blue), while the cells treated with a combination of Fe²⁺ and Artesunate showed much more blue-colored dead cells (and dead cell clusters). These results reveal that Artesunate exhibits low basal cytotoxicity against B16-F10 cells and Artesunate cytotoxicity is enhanced in the presence of additional extracellular Fe⁺. FIG. 8 shows microscopic photographs of B16-F10 cells stained with Trypan blue; (a) untreated cells, (b) cells treated with 4 μM Fe2+ for 24 h, (c) cells treated with 200 μM Artesunate for 24 h, (d) cells treated with 4 μM Fe2+ and 200 μM Artesunate for 24 h.

To explore the cytotoxicity of Artesunate against other types of cancer cells and role of extracellular Fe2+ concentration, human HCT116 colon cancer cells were incubated with 20 μM Artesunate and 0 or 4 μM extracellular Fe2+ for 24 hours. As seen from the microscopic photographs of Trypan stained cells in FIG. 9, Artesunate-treated cells showed a significant fraction of dead cells and cells treated with a combination of Fe²⁺ and Artesunate showed a higher fraction of dead cells. These results reveal that Artesunate exhibits low basal cytotoxicity against HCT-116 cells and Artesunate cytotoxicity is enhanced in the presence of additional extracellular Fe². FIG. 9 shows microscopic photographs of HCT116 cells stained with trypan blue; (a) untreated cells, (b) cells treated with 4 μM Fe2+ for 24 h, (c) cells treated with 20 μM Artesunate for 24 h, (d) cells treated with 4 μM Fe2+ and 20 μM Artesunate for 24 h.

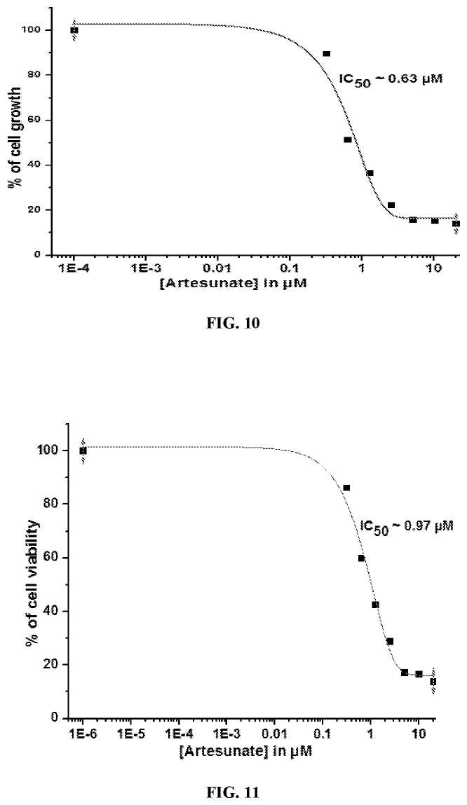

To quantify the dose-dependent cytotoxicity of Artesunate against HCT116 cancer cells, a sulforhodamine B (SRB) assay was performed. A dose-dependent inhibition of the proliferation of HCT116 cells by Artesunate was observed with IC₅₀ estimated to be ˜0.63 μM. FIG. 10 shows the dose-response results of Artesunate toxicity against HCT116 cells measured by SRB colorimetric assay.

### Example 4. Cytotoxicity of Artesunate and ART-HER2 Conjugate Against HER2-Positive Cancer Cells (Caco-2 cell line) and HER2-Negative Normal Cells

Experiments were performed to determine if the ART-HER2 conjugate (described in Example 1) exhibited increased cytotoxicity against HER2-positive cancer cells compared to the cytotoxicity of artensunate alone against HER2-positive cancer cells and compared to the cytotoxicity of ART-HER2 against HER2-negative cells.

### Materials and Methods

### Cell Culture

CCD-18 cells were maintained in Dulbecco's minimal essential medium (DMEM, ATCC) supplemented with 10% fetal bovine serum (FBS, ATCC), 100 U/ml penicillin G, and 100 μg/ml streptomycin at 37° C. in a humid atmosphere of 5% CO2 atmosphere. Caco-2 cells were maintained in Dulbecco's minimal essential medium (DMEM, ATCC) supplemented with 20% fetal bovine serum (FBS,ATCC), 100 U/ml penicillin G, and 100 μg/ml streptomycin at 37° C. in a humid atmosphere of 5% CO2 atmosphere; culture medium was replaced with a fresh medium every 2-3 days. All experiments were performed with actively growing cells in logarithmic growth phase.

### MTT Assay

The assay was carried out following a reported procedure [45]. In brief, approximately 1,000 to 5,000 cells were plated per well in 96-well plate. The plates were incubated at 37° C. for 48 or 72 hours in a humidified, 5% CO2 atmosphere. Cells were incubated either with Artesunate or with ART-HER2 conjugate at various concentrations. To measure mitochondrial activity, a metric for cell viability, 15 μL of [3-{4, 5-dimethylthiazol-2-yl)-2, 5-diphenyltetrazolium bromide (MTT) was added to each well and incubated at 37° C. for 4 hours in a humidified, 5% CO2 atmosphere. After incubation, 100 μl of the solubilization solution/stop mix was added to each well. The blue formazan product formed by reduction of MTT by mitochondrial dehydrogenase in viable cells was then measured by absorbance at 570 nm [44].

### Results

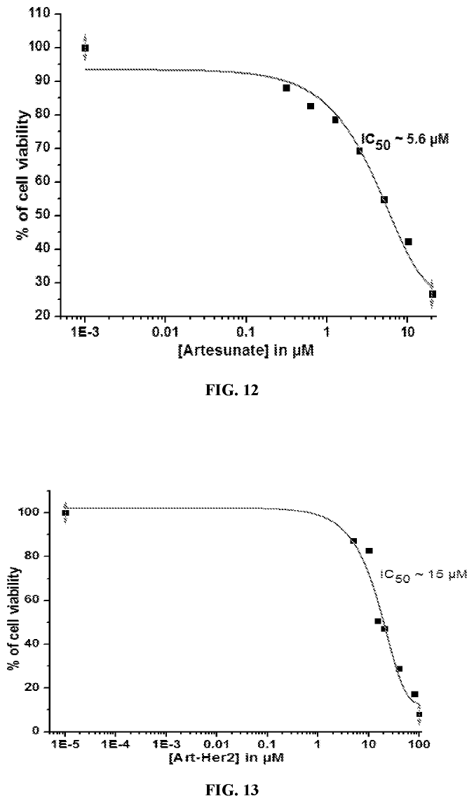

The cytotoxicity of Artesunate was then investigated with two human colon cell lines, a normal colon cell line CCD-18 and a cancerous colon cell line Caco-2, using the well-established MTT assay [42]. Notably, CCD-18 cells are HER2-negative and Caco-2 cells are HER2-positive. Dose-dependent assays were performed in 96-well format and IC50 values were determined by plotting the cell viability against Artesunate concentration (FIG. 11 and FIG. 12). In particular, FIG. 11 shows the dose response analysis of Artesunate toxicity against Caco-2 cell lines using the MTT assay. FIG. 12 shows the dose response analysis of Artesunate toxicity against CCD-18 cell lines using the MTT assay in a 96-well format. Artesunate displayed high cytotoxicity against both the normal and the cancerous colon cells with IC50 values in micromolar range: IC50 ˜5.6 μM for non-cancerous CCD-18 cells and IC 50 ˜0.97 μM for cancerous Caco-2 cells. Notably, Artesunate displays nearly 5.7 fold higher toxicity against Caco-2 cells compared to CCD-18 cells.

For target-specific cell killing, it is critically required for Art-Her2 conjugate to specifically bind to the HER2 receptor on the cell surface. Cancerous Caco-2 cells are known to over-express the Her2 receptor while the normal CCD-18 does not [32]. Artesunate is membrane permeable, thus it can be taken up by all cell types. However, the Art-Her2 conjugate is not membrane permeable and is expected to be taken up only by cells that express Her2 receptors. It is thus interesting to compare the cytotoxicity of the Art-Her2 conjugate in normal CCD-18 cells and cancerous Caco-2 cells for testing the proposed targeted anticancer drug development using the Art-Her2 conjugate. MTT assays were performed using 96-well plate with normal CCD-18 cells and cancerous Caco-2 cells.

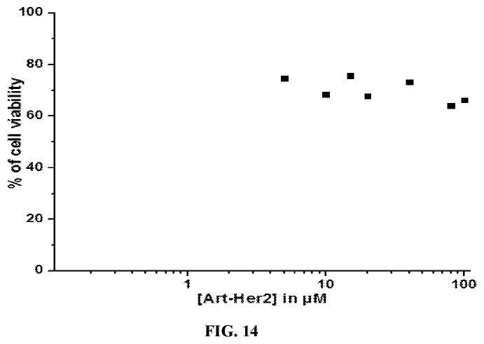

As shown in FIG. 13 (which shows the dose response analysis of Art-Her2 conjugate toxicity against Caco-2 cell lines using the MTT assay in a 96-well format) and FIG. 14 (which shows the dose response analysis of Art-Her2 conjugate toxicity against CCD-18 cell lines using the MTT assay in a 96-well format), the Art-Her2 conjugate showed a well-behaved dose-response curve and high cytotoxicity (or anti-proliferative effect) against Caco-2 cells with IC 50 in micromolar level (˜15 μM). In contrast, the dose-dependent profile with normal CCD-18 cells showed none-response to increased concentration of Art-Her2, with similar high viability at all the Art-Her2 concentrations tested (maximum 100 μM; IC50 could not be determined from this experiment). Art-Her2 conjugate thus has a greatly reduced cytotoxicity against the normal CCD-18 cells. This greatly reduced cytotoxicity may due to that the Art-Her2 conjugate may not be taken up by the normal CCD-18 cells, which do not express Her2 receptor.

The presented assays examining viability of cell lines cultured in presence of increasing concentrations of ART-HER2 conjugate reveal that our ART-HER2 conjugate exhibits selective cytotoxicity against HER2-positive cells, the Caco-2 cells, with IC50 ˜15 μM and no measurable cytotoxicity against HER2-negative cells even at the highest ART-HER2 conjugate levels tested at 100 μM. The cytotoxicity of Artesunate against HER2-positive cells, the Caco-2 cells, is higher (IC50 values ˜0.97 μM) than the cytotoxicity of our ART-HER2 conjugate (IC50 ˜15 μM), suggesting that our ART-HER2 conjugate, while specific against HER-2 positive cancer cells, is less cytotoxic than Artesunate alone. This is reasonable considering that the larger size of Art-Her2, which prevent it from passing through the cell membrane freely. The receptor-mediated cellular uptake of Art-Her2 is less efficient than the diffuse route of the free artesunate. However, the much lowered cytotoxicity to normal cells is an important and a much desired feature for such a targeted drug delivery strategy. In addition, the anticancer ability of Art-Her2 conjugate (IC 50 ˜15 μM) is still comparable to the commonly used anticancer drugs such as 5-FU (IC 50˜15 μM), carboplatin (IC 50˜12 μM) [50] which are the 1st-line drugs that are currently used for the treatment of colon cancers. Toxic side effects are a common problem of these non-targeted anticancer drugs. These interesting features suggested that the Art-HER2 conjugate may have the ability to effectively deliver the cytotoxic artesunate molecule specifically to Her2 positive cancerous cells thus is a promising strategy for new targeted anti-cancer drug development.

### Example 5. Quantification of Intracellular Fe²⁺ in HER2-Positive Cancer Cells (Caco-2 cell line) and HER2-Negative Normal Cells

As the in vitro results of 2-deoxyribose degradation assay and dichlorofluorescein (DCF) assay demonstrated that artesunate can promote Fe^{2 +} depended ROS generation, which may be responsible to its cytotoxicity; and it has been postulated that cancer cells may have a higher free iron level than normal cells [49], it is thus hypothesized that the higher cytotoxicity of Artesunate against Caco-2 cells might be due to a higher free iron level in the Coao-2 cancer cells compared to that of in the normal CCD-18 cells. However, the cellular free iron levels in these cells have not been determined. To test this hypothesis, the free iron levels in these cells were determined using a ratiometric iron imaging sensor.

### Materials and Methods

### Cell Culture

The concentrations of chelatable Fe²⁺ pools in human normal colon (CCD-18) and human epithelial colorectal adenocarcinoma (Caco-2) cell lines were determined. CCD-18 cells were maintained in Dulbecco's minimal essential medium (DMEM, ATCC) supplemented with 10% fetal bovine serum (FBS, ATCC), 100 U/ml penicillin G, and 100 μg/ml streptomycin at 37° C. in a humid atmosphere of 5% CO2 atmosphere. Caco-2 cells were maintained in Dulbecco's minimal essential medium (DMEM, ATCC) supplemented with 20% fetal bovine serum (FBS, ATCC), 100 U/ml penicillin G, and 100 μg/ml streptomycin at 37° C. in a humid atmosphere of 5% CO2 atmosphere; culture medium was replaced with a fresh medium every 2-3 days. After being nearly confluent, the cells were used for experiments.

### Confocal Microscopic Imaging

A Zeiss LSM 710 laser-scanning confocal microscope system was used for cell imaging experiments. 40× oil-immersion objective lens were used to perform all the experiments. Subcellular organelles mitochondria and lysosomes were imaged with MitoTracker® Green FM and LysoTracker® Red DND-100, respectively, with excitation wavelengths recommended by the manufacturer were 488 nm for MitoTracker®, 543 nm for LysoTracker®. Emissions were integrated at 492-535 nm (MitoTracker®), 550-625 nm (LysoTracker®), respectively. Fe⁺ levels in cells were determined using a ratiometric Fe⁺ sensor NIRh-Fret with excitation wavelengths of the laser at 405 nm and 633 nm and emissions were collected over the range 420-700 nm and 650-850 nm, respectively.

### Results

To determine if the enhanced cytotoxicity of ART-HER2 conjugate against HER2-positive cells compared to HER2-negative cells correlates with intracellular Fe²⁺ concentration, the intracellular concentrations of Fe⁺ were determined using in the Caco-2 and CCD-18 cells grown in culture. Specifically a Fe⁺ specific ratiometric sensor NIRh-Fret was applied to cells grown in culture and the sensor was visualized by confocal microscopy. The Fe⁺ intracellular concentrations were determined to be ˜12±1 μM in the mitochondria and ˜9±1 μM in the lysosomes of Caco-2 cells. The Fe⁺ intracellular concentrations for CCD-18 cells were determined to be ˜7±1 μM in the mitochondria and ˜5±1 μM in the lysosomes. These results show that the Fe²⁺ intracellular concentrations are higher in HER2-positive Caco-2 cells compared to HER2-negative CCD-18 cells. This significantly higher free Fe²⁺ levels in Caco-2 cells (71% higher in mitochondria and 80% higher in lysosomes) correlate well with its higher ART-HER2 conjugate cytotoxicity against Caco-2 cells compared to CCD-18 cells grown in culture measured in Example 4. Based on these results, the ART-HER2 conjugate is predicted to exhibit higher cytotoxicity against HER2-positive cells with elevated intracellular Fe⁺ concentration.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1. Ginghaosu Antimalaria Coordinating Research Group, Antimalaria studies on qinghaosu, Chin Med J., 1979, 92, 811.
2. D. L. Klayman, Qinghaosu (artemisinin): an antimalarial drug from china, Science, 1985, 228, 1049.
3. S. R. Meshnick, T. W. Tsang, F. B. Lin, H. Z. Pan, C. N. Chang, F. Kuypers, D. Chiu, B. Lubin B. Activated oxygen mediates the antimalarial activity of qinghaosu, Prog. Clin Biol. Res., 1989, 313, 95.
4. S. R. Meshnick, Y. Z. Yang, V. Lima, F. Kuypers, S. Kamchonwongpaisan, Y. Yuthavong, Iron-dependent free radical generation from the antimalarial agent artemisinin, Antimicrob Agents Chemother, 1993. 37, 1108.
5. F. Zhang, D. K.Gosser, S. R. Meshnick, Hemin-cataluzed decomposition of artemisinin(qinghaosu), Biochem Pharmacol., 1992, 43, 1805.
6. G. H. Posner, J. N. Cumming, M. Krasavin, Carbon Centered Radicals, John Wiley & Sons, River Street, New York, 2000.
7. K. M. Anderson, T. Seed, D. Ou, J. E. Harris, Free radicals and reactive oxygen species in programmed cell death, Med Hypotheses, 1999, 52, 451.
8. A. Benakis, M. Paris, L. Loutan, C. T. Plessas, S. T. Plassas, Pharmacokinetics of artemisinin and artesunate after oral administration in healthy volunteers, Am J Trop Med Hyg., 1997, 56, 17.
9. J. Fishwick, W. G. McLean, G. Edwards, S. A. Ward, The toxicity of artemisinin and related compounds on neuronal and glial cells in culture, Chem Biol Interact., 1995, 96, 263.
10. T. T. Hien, N. J. White, Qinghaosu, Artemisinin compounds in treatment of malaria, Lancet, 1993, 341, 603.
11. S. Kamchonwongpaisan, P. McKeever, P. Hossler, H. Ziffer, S. R. Meshnick, Artemisinin neurotoxicity: neuropathology in rats and mechanistic studies in vitro, Am J Trop Med Hy., 1997, 56, 7.
12. G. R. Greenberg, M. M. Wintrare, Manganese superoxide dismutase depletion in murine hematopoietic stem cells perturbs iron homeostasis, globin switching and epigenetic control in erythrocyte precursor cells, J. Biol. Chem., 1946, 165, 397.
13. I. Nakase, H. Lai, N. P. Singh, T. Sasaki, Anticancer properties of artemisinin derivatives and their targeted delivery by transferrin conjugation, Int J Pharm., 2008, 354, 28.
14. P. Veerasubramanian, P. Gosi, C. Limsomwong, D. S. Walsh, Artesunate and a major metabolite, dihydroartemisinin, diminish mitogen-induced lymphocyte proliferation and activation, J Trop Med Public Health., 2006, 37, 838.
15. M. Wrona, K. Patel, P. Wardman, Reactivity of 2′,7′-dichlorodihydrofluorescein and dihydrorhodamine 123 and their oxidized forms toward carbonate, nitrogen dioxide and hydroxyl radicals, Free Radical Biol. Med., 2005, 38, 262.
16. T. Efferth, H. Dunstan, A. Sauerbrey, H. Miyachi, C. R. Chitambar, The antimalarial artesunate is also active against cancer, Int J Oncol., 2001, 18, 767.
17. X. J. Huang, Z. Q. Ma, W. P. Zhang, Y. B. Lu, E. Q. Wei, Dihydroartemisinin exerts cytotoxic effects and inhibits hypoxia inducible factor-lalpha activation in C6 glioma cells, J Pharm Pharmacol., 2007, 59, 849.
18. Y. Jiao, C. M. Ge, Q. H. Meng, J. P. Cao, J. Tong, S. J. Fan, Dihydroartemisinin is an inhibitor of ovarian cancer cell growth, Acta Pharmacol. Sin., 2007, 28, 1045.
19. R. Dell'Eva, U. Pfeffer, R. Vene, L. Anfosso, A. Forlani, A. Albini, T. Efferth, Inhibition of angiogenesis in vivo and growth of kaposi's sarcoma xenograft tumors by the antimalarial artesunate, Biochem. Pharmacol., 2004, 68, 2359.
20. M. Karin, B. Mintz, Receptor-mediated endocytosis of transferrin in developmentally totipotent mouse teratocarcinoma stem cells, J. Biol. Chem., 1981, 256, 3245.
21. N. Shterman, B. Kupfer, C. Moroz, Comparison of transferrin receptors, iron content and isoferritin profile in normal and malignant human breast cell lines, Pathobiol., 1991, 59, 19.
22. M. Karin, B. Mintz, Receptor-mediated endocytosis of transferrin in developmentally totipotent mouse teratocarcinoma stem cells, J. Biol. Chem., 1981, 256, 3245.
23. M. Wrona, K. Patel, P. Wardman, Reactivity of 2′,7′-dichlorodihydrofluorescein and dihydrorhodamine 123 and their oxidized forms toward carbonate, nitrogen dioxide and hydroxyl radicals, Free Radical Biol. Med., 2005, 38, 262.
24. W. S. May, P. Cuatrecasas, Transferrin receptor: its biological significance, J Membrane Biol., 1985, 88, 205.
25. N. P. Singh, H. C. Lai, Artemisinin induces apoptosis in human cancer cells, Anticancer Res., 2004, 24, 2277.
26. W. Nam, J. Tak, J. K. Ryu, Effects of artemisinin and its derivatives on growth inhibition and apoptosis of oral cancer cells, Head Neck., 2007, 29, 335.
27. P. M. O'Neill, G. H. Posner, A medicinal chemistry perspective on artemisinin and related endoperoxides, J. Med. Chem., 2004, 47, 2945.
28. J. C. Kwok, D. R. Richardson, The iron metabolism of neoplastic cells: alterations that facilitate proliferation, Crit. Rev. Oncol. Hematol., 2002, 42, 65.
29. S. M. Zhang, G. S. Gerhard, Heme mediates cytotoxicity from artemisinin and serves as a general anti-proliferation target, PLoS One., 2009, 4, 7472.
30. T. Efferth, A. Sauerbrey, A. Olbrich, E. Gebhart, P. Rauch, H. O. Weber, H. J. G. Hengstler, M. E. Halatsch, M. Volm, K. D. Tew, D. D. Ross, J. O. Funk, Molecular modes of action of artesunate in tumor cell lines, Mol. Pharmacol., 2003, 64, 382.
31. G. D. Wu, H. J. Zhou, X. H.Wu, Apoptosis of human umbilical vein endothelial cells enduced by artesunate, Vascul. Pharmacol., 2004, 41, 205.
32. I. Rubin, Y. Yarden, The basic biology of HER2, Ann Oncol., 2001, 12, 8.
33. N. I. Pollock and J. R. Grandis, HER2 as a Therapeutic Target in Head and Neck Squamous Cell Carcinoma, Clin Cancer Res; 2014, 21(3); 526-33.
34. K. Ghaffarzadegan, N. Sharifi, H. Vosooghynia, T. G. Moghadam, S. G. Kafi, G. Nassiri, HER2/neu expression in colon adenocarcinoma and its correlation with clinicopathologic variables, IJBMS, 2006, 9, 64.
35. D. J. Slamon, B. L. Jones, S. Shak, H. Fuchs, V. Paton, A. Bajamonde, Use of chemotherapy plus a monoclonal antibody against HER2 for metastatic breast cancer that overexpresses HER2, N Engl J Med., 2001, 344, 783.
36. K. L. Blackwell, H. J. Burstein, A. M. Storniolo, H. Rugo, G. Sledge, M. Koehler, Randomized study of lapatinib alone or in combination with trastuzumab in women with ERBB2 positive, trastuzumab-refractory metastatic breast cancer, J Clin Oncol., 2010, 28, 1124.
37. C. E. Geyer, J. Forster, D. Lindquist, S. Chan, C. G. Romieu, T. Pienkowski T, Lapatinib plus capecitabine for HER2 positive advanced breast cancer, N Engl J Med., 2006, 355, 2733.
38. (a) A. D. Leo, H. L. Gomez, Z. Aziz, Z. Zvirbule, J. Bines, M. C. Arbushites, Phase III, double-blind, randomized study omparing lapatinib plus paclitaxel with placebo plus paclitaxel as first-line treatement for metastatic breast cancer, J Clin Oncol., 2008, 26, 5544.
   (b) Kaifee Arman, Sercan Ergün, Ebru Temiz, Serdar Öztuzcu, Deciphering the role of ErbB2/HER2 in cancer cell lines: a proto-oncogene with antiapoptotic activity, Cancer Cell & Microenvironment 2014; 1: e448. doi: 10.14800/ccm.448;
39. J. M. Gutteridge, B. Halliwell, The deoxyribose assay: an assay both for free hydroxyl radical and for site-specifichydroxyl radical production, Biochem J., 1988, 253, 932.
40. C. A. Perez, Y. Wei, M. Guo, Iron-binding and anti-fenton properties of baicalin, J. Inorg. Biochem., 2009, 103, 326.
41. V. Vichai, K. Kirtikara, Sulforhodamine B colorimetric assay for cytotoxicity screening, Nature Protocols., 2006, 1, 1112.
42. Y. Tampo, S. Kotamraju, C. R. Chitambar, S. V. Kalivendi, A. Keszler, J. Joseph, B. Kalyanaraman, Unraveling the biological roles of reactive oxygen species, Circ Res., 2011, 13, 361.
43. C. P. Lebel, H. Ischiropoulos, S. C. Bondys, Evaluation of the probe2′, 7′-dichlorofluorescin as an indicator of reactive oxygen species formation and oxidative stress, Chem. Res. Toxicol., 1992, 5, 227.
44. T. Bemas, J. Dobrucki, Mitochondrial and nonmitochondrial reduction of MTT: interaction of MTT with TMRE, JC-1 and NAO mitochondrial fluorescent probes, Cytometry, 2002, 47, 236.
45. J. Carmichael, W. G. Degraff, A. F. Gazdar, J. D. Minna, J. B. Mitchell, Evaluation of a tetrazolium-based semiautomated colorimetric assay: assessment of chemosensitivity testing, Cancer Res., 1987, 47, 936.
46. L. Wang, M. Switalska, N. Wang, Z. J. Du, Y. Fukumoto, N. K Diep, R. Kiguchi, J. Nokami, J. Wietrzyk, T. Inokuchi, Design, synthesis and biological evaluation of artemisinin indoloquinoline hybrids as potent antiprolifeferative agents, Molecules., 2014, 19, 19021.
47. P. Skehan, R. Storeng, D. Scudiero, A. Monks, J. McMahon, D. Vistica, J. T. Warren, H. Bokesch, S. Kenney, M. R. Boyd, New colormetric cytotoxicity assay for anticancer-drug screening, J. Natl Cancer Inst., 1990, 82, 1107.
48. R. H. J. Lillie, Conn's Biological Stains 9 th edn, Williams & Wilkins, Wilkins Street, Baltimore, 1977.
49. J. C. Kwok, D. R. Richardson, The iron metabolism of neoplastic cells: alterations that facilitate proliferation, The iron metabolism of neoplastic cells: alterations that facilitate proliferation, Crit. Rev. Oncol. Hematol., 2002, 42, 65.
50. M. F. Sichani, S. Honarnejad, L. M. Heiser, J. W. Gray, P. K. Sorger, Metrics other than potency reveal systematic variation in responses to cancer drugs, Nat. Chem. Biol., 2013, 9, 708.

1. A composition comprising
a Her2 peptide that targets the Her2 receptor and comprises amino acid sequence GSGKCCYSL (SEQ ID NO:1); and
a cytotoxic agent comprising artemisinin (Art) or a derivative thereof linked to the peptide.

2. The composition of claim 1, wherein the cytotoxic agent comprises one or more of artesunate and dihydroartemisinin.

3. The composition of claim 1, wherein the Her2 peptide and cytotoxic agent are chemically linked via peptide conjugation chemistry.

4. The composition of claim 1, comprising two or more Her2 peptides.

5. The composition of claim 1, wherein an amine functional group of an N-terminus of the Her2 peptide is chemically linked to a carbonyl group of artemisinin or derivative thereof.

6. The composition of claim 5, wherein the artemisinin derivative is artesunate.

7. The composition of claim 1, further comprising one or more supplementary active agents selected from the group consisting of adriamycin, cyclophosphamide, taxotere, vinblastine, dacarbazine, etoposide, vincristine, procarbazine, predniscone, cisplatin, 5-fluorouracil, and gemcitabine.

8-9. (canceled)

10. A method of inhibiting growth of Her2-positive cells in a subject, the method comprising:
identifying a subject with Her2-positive cells; and
administering to the subject an effective amount of a composition comprising
a Her2 peptide that targets the Her2 receptor and comprises amino acid sequence GSGKCCYSL (SEQ ID NO:1); and
a cytotoxic agent linked to the peptide, wherein the cytotoxic agent comprises artemisinin (Art) or a derivative thereof.

11. The method of claim 10, wherein the cytotoxic agent comprises one or more of artesunate and dihydroartemisinin.

12. The method of claim 10, wherein the composition further comprises one or more supplementary active agents selected from the group consisting of adriamycin, cyclophosphamide, taxotere, vinblastine, dacarbazine, etoposide, vincristine, procarbazine, predniscone, cisplatin, 5-fluorouracil, or gemcitabine.

13. The method of claim 10, wherein the Her2-positive cancer is selected from the group consisting of breast, lung, liver, colon, prostate, bladder, cervix, endometrium, germ cell, glioblastoma, head and neck, ovarian, pancreas, salivary duct, or gastric cancer.
